(19) United States
(12) Patent Application Publication
(10) Pub. No.: US 2009/0035855 A1
(43) Pub. Date: Feb. 5, 2009

(54) THREE-DIMENSIONAL RECONSTITUTED EXTRACELLULAR MATRICES AS SCAFFOLDS FOR TISSUE ENGINEERING

(75) Inventors: Jackie Y. Ying, Singapore (SG); Kwong Joo Leck, Singapore (SG); Andrew C.A. Wan, Singapore (SG)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Agency for Science, Technology and Research, Singapore (SG)

(21) Appl. No.: 12/095,486

(22) PCT Filed: Dec. 1, 2006
(86) PCT No.: PCT/SG2006/000376

§ 371 Date: Aug. 27, 2008

Related U.S. Application Data
(60) Provisional application No. 60/741,370, filed on Dec. 1, 2005.

(51) Int. Cl. C12N 5/06

(52) U.S. Cl. 435/377; 435/395; 435/375

(57) ABSTRACT

A biomaterial scaffold comprising: a) reconstituted extracellular matrix; and b) polyelectrolyte complex fibers; wherein the matrix and the fibers are functionally associated.

### FIELD OF THE INVENTION

The present invention relates to fibrous scaffolds comprising extracellular matrix and to their use in tissue engineering.

### BACKGROUND OF THE INVENTION

Tissue engineering (TE) is an interdisciplinary field that applies the principles of engineering and life sciences toward the development of biological substitutes that restore, maintain, or improve tissue function of a whole organ. Tissue engineering techniques and materials find increasing application in a wide range of therapeutic and clinical procedures including but not limited to tissue grafts and organ transplants. Tissue engineering is still an emergent field characterized by considerable knowledge gaps.

Key knowledge gaps in the late 1980's and early 1990's included sources of large quantities of cells reliably and controllably expressing desired phenotypes, details of the immune response to implanted tissues, the role of chemical and physical signals in morphogenesis and in the in vivo remodeling of implanted tissues, means of controlling angiogenesis in order to achieve adequate vascularization of three-dimensional tissue constructs, design principles to create and optimize bioreactors and bioprocessing techniques for the manufacture of specific tissue-engineered products and means of preserving TE products between the point of manufacture and the time of usage.

Tissue engineering typically uses living cells as engineering materials. Cells to be used in the process of tissue engineering are transplanted onto a scaffold. A scaffold may be conveniently defined as any artificial structure which allows for three-dimensional tissue formation.

Desirable characteristics for a scaffold include but are not limited to adaptation for cell attachment and diffusion of cell nutrients and expressed products. The proper diffusion of cell nutrients is required for the development of the tissue on the scaffold. Biodegradability is another desirable characteristic for a scaffold due to the fact that surgical removal of a scaffold would generally be required in the event that the scaffold is not absorbed by the surrounding tissue.

For both tissue development and regeneration, a myriad of factors contribute to the growth and differentiation of cells to form tissues. These factors must be presented on the biomaterial matrices or scaffolds that are employed for tissue engineering¹, in a manner whereby they are accessible to the cells.

There are several problems associated with the provision of nutrients to cells developing on scaffolds. The use of certain nutrient materials such as recombinant factors is limited by the extremely high costs associated with such products. Furthermore there are broad gaps in the level of knowledge regarding the factors involved in regeneration, strategies for immobilizing bioactive ligands on the scaffold or delivering biomolecules in a sustained fashion from the scaffolds. This knowledge gap has resulted in solutions that usually focus on a minute fraction of the total spectrum of biological activity that a scaffold can potentially be endowed with.

Therefore there is a need for improved techniques of delivering cell nutrients to cells on a scaffold. In particular, there is a need for improved techniques of delivering a wide range of nutrients to cells on a scaffold.

There is the further need for methods to increase the range of biological activity for a given scaffold.

### SUMMARY OF THE INVENTION

The present invention relates to the incorporation of extracellular matrix, secreted by cells in culture or derived from animal tissue, into fibers formed by interfacial polyelectrolyte complexation forming the basis by which the ECM can be reconstituted to form three dimensional scaffolds. Such 3D matrices are useful to investigate the influence of the ECM on cell phenotype, and constitutes a promising approach to the engineering of functional tissue.

According to a first aspect of the present invention, there is provided a biomaterial scaffold comprising reconstituted extracellular matrix; and polyelectrolyte complex fibers wherein the matrix and the fibers are functionally associated.

According to one embodiment of the first aspect, the polyelectrolyte complex fibers are comprised of a polycation precursor and a polyanion precursor. In another embodiment, the polycation precursor is chitosan and the polyanion precursor is sodium alginate. In another embodiment, reconstituted extracellular matrix is incorporated into the polycation precursor and the polyanion precursor.

In a further embodiment, reconstituted extracellular matrix is incorporated into the polycation precursor or the polyanion precursor. In yet a further embodiment, reconstituted extracellular matrix is incorporated into the polyanion precursor. In yet a further embodiment, the reconstituted extracellular matrix is derived from cultured cells or animal tissue. In yet a further embodiment, the animal tissue is selected from the group comprising skin, liver, pancreas, kidney, bone marrow, muscle, heart, lungs, gastro-intestinal tract, brain and small intestinal submucosa. The animal tissue may be rat liver tissue.

In an embodiment of the first aspect, the reconstituted extracellular matrix is derived from cell culture or cells selected from any one of the group comprising embryonic stem cells, adult stem cells, blast cells, cloned cells, placental cells, keratinocytes, basal epidermal cells, urinary epithelial cells, salivary gland cells, mucous cells, serous cells, von Ebner's gland cells, mammary gland cells, lacrimal gland cells, ceruminous gland cells, eccrine sweat gland cells, apocrine sweat gland cells, Moll gland cells, sebaceous gland cells, Bowman's gland cells, Brunner's gland cells, seminal vesicle cells, prostate gland cells, bulbourethral gland cells, Bartholin's gland cells, Littre gland cells, uterine endometrial cells, goblet cells of the respiratory or digestive tracts, mucous cells of the stomach, zymogenic cells of the gastric gland, oxyntic cells of the gastric gland, insulin-producing β cells, glucagon-producing a cells, somatostatin-producing. DELTA cells, pancreatic polypeptide-producing cells, pancreatic ductal cells, Paneth cells of the small intestine, type II pneumocytes of the lung, Clara cells of the lung, anterior pituitary cells, intermediate pituitary cells, posterior pituitary cells, hormone secreting cells of the gut or respiratory tract, thyroid gland cells, parathyroid gland cells, adrenal gland cells, gonad cells, juxtaglomerular cells of the kidney, macula densa cells of the kidney, peri polar cells of the kidney, mesangial cells of the kidney, brush border cells of the intestine, striated ducted cells of exocrine glands, gall bladder epithelial cells, brush border cells of the proximal tubule of the kidney, distal tubule cells of the kidney, conciliated cells of the ductulus efferens, epididymal principal cells, epididymal basal cells, hepatocytes, fat cells, type I pneumocytes, pancreatic duct cells, nonstriated duct cells of the sweat gland, nonstriated duct cells of the salivary gland, nonstriated duct cells of the mammary gland, parietal cells of the kidney glomerulus, podocytes of the kidney glomerulus, cells of the thin segment of the loop of Henle, collecting duct cells, duct cells of the seminal vesicle, duct cells of the prostate gland, vascular endothelial cells, synovial cells, serosal cells, squamous cells lining the perilymphatic space of the ear, cells lining the endolymphatic space of the ear, choroid plexus cells, squamous tells of the pia-arachnoid, ciliary epithelial cells of the eye, corneal endothelial cells, ciliated cells having propulsive function, ameloblasts, planum semilunatum cells of the vestibular apparatus of the ear, interdental cells of the organ of Corti, fibroblasts, pericytes of blood capillaries, nucleus pulposus cells of the intervertebral disc, cementoblasts, cementocytes, odontoblasts, odontocytes, chondrocytes, osteoblasts, osteocytes, osteoprogenitor cells, hyalocytes of the vitreous body of the eye, stellate cells of the perilymphatic space of the ear, skeletal muscle cells, heart muscle cells, smooth muscle cells, myoepithelial cells, red blood cells, platelets, megakaryocytes, monocytes, connective tissue macrophages, Langerhan's cells, osteoclasts, dendritic cells, microglial cells, neutrophils, eosinophils, basophils, mast cells, plasma cells, helper T cells, suppressor T cells, killer T cells, killer cells, rod cells, cone cells, inner hair cells of the organ of Corti, outer hair cells of the organ of Corti, type I hair cells of the vestibular apparatus of the ear, type II cells of the vestibular apparatus of the ear, type II taste bud cells, olfactory neurons, basal cells of olfactory epithelium, type I carotid body cells, type II carotid body cells, Merkel cells, primary sensory neurons specialised for touch, primary sensory neurons specialised for temperature, primary neurons specialised for pain, proprioceptive primary sensory neurons, cholinergic neurons of the autonomic nervous system, adrenergic neurons of the autonomic nervous system, peptidergic neurons of the autonomic nervous system, inner pillar cells of the organ of Corti, outer pillar cells of the organ of Corti, inner phalangeal cells of the organ of Corti, outer phalangeal cells of the organ of Corti, border cells, Hensen cells, supporting cells of the vestibular apparatus, supporting cells of the taste bud, supporting cells of the olfactory epithelium, Schwann cells, satellite cells, enteric glial cells, neurons of the central nervous system, astrocytes of the central nervous system, oligodendrocytes of the central nervous system, anterior lens epithelial cells, lens fibre cells, melanocytes, retinal pigmented epithelial cells, iris pigment epithelial cells, oogonium, oocytes, spermatocytes, spermatogonium, ovarian follicle cells, Sertoli cells, and thymus epithelial cells, hepatocarcinoma or combinations thereof.

The reconstituted extracellular matrix may be derived from an osteoblast cell line or a hepatocarcinoma cell line. The osteoblast cell line is MC-3T3. The hepatocarcinoma cell line is HepG2. The biomaterial scaffold may further comprise at least one stabilising agent.

In another embodiment of the first aspect, the biomaterial scaffold may further comprise at least one biologically active agent, and wherein the biologically active agent comprises a plurality of cells seeded within the polyelectrolyte complex fibers.

The plurality of cells are selected from any one of the group comprising embryonic stem cells, adult stem cells, blast cells, cloned cells, placental cells, keratinocytes, basal epidermal cells, urinary epithelial cells, salivary gland cells, mucous cells, serous cells, von Ebner's gland cells, mammary gland cells, lacrimal gland cells, ceruminous gland cells, eccrine sweat gland cells, apocrine sweat gland cells, Moll gland cells, sebaceous gland cells, Bowman's gland cells, Brunner's gland cells, seminal vesicle cells, prostate gland cells, bulbourethral gland cells, Bartholin's gland cells, Littre gland cells, uterine endometrial cells, goblet cells of the respiratory or digestive tracts, mucous cells of the stomach, zymogenic cells of the gastric gland, oxyntic cells of the gastric gland, insulin-producing β cells, glucagon-producing a cells, somatostatin-producing DELTA cells, pancreatic polypeptide-producing cells, pancreatic ductal cells, Paneth cells of the small intestine, type II pneumocytes of the lung, Clara cells of the lung, anterior pituitary cells, intermediate pituitary cells, posterior pituitary cells, hormone secreting cells of the gut or respiratory tract, thyroid gland cells, parathyroid gland cells, adrenal gland cells, gonad cells, juxtaglomerular cells of the kidney, macula densa cells of the kidney, peri polar cells of the kidney, mesangial cells of the kidney, brush border cells of the intestine, striated ducted cells of exocrine glands, gall bladder epithelial cells, brush border cells of the proximal tubule of the kidney, distal tubule cells of the kidney, conciliated cells of the ductulus efferens, epididymal principal cells, epididymal basal cells, hepatocytes, fat cells, type I pneumocytes, pancreatic duct cells, nonstriated duct cells of the sweat gland, nonstriated duct cells of the salivary gland, nonstriated duct cells of the mammary gland, parietal cells of the kidney glomerulus, podocytes of the kidney glomerulus, cells of the thin segment of the loop of Henle, collecting duct cells, duct cells of the seminal vesicle, duct cells of the prostate gland, vascular endothelial cells, synovial cells, serosal cells, squamous cells lining the perilymphatic space of the ear, cells lining the endolymphatic space of the ear, choroid plexus cells, squamous cells of the pia-arachnoid, ciliary epithelial cells of the eye, corneal endothelial cells, ciliated cells having propulsive function, ameloblasts, planum semilunatum cells of the vestibular apparatus of the ear, interdental cells of the organ of Corti, fibroblasts, pericytes of blood capillaries, nucleus pulposus cells of the intervertebral disc, cementoblasts, cementocytes, odontoblasts, odontocytes, chondrocytes, osteoblasts, osteocytes, osteoprogenitor cells, hyalocytes of the vitreous body of the eye, stellate cells of the perilymphatic space of the ear, skeletal muscle cells, heart muscle cells, smooth muscle cells, myoepithelial cells, red blood cells, platelets, megakaryocytes, monocytes, connective tissue macrophages, Langerhan's cells, osteoclasts, dendritic cells, microglial cells, neutrophils, eosinophils, basophils, mast cells, plasma cells, helper T cells, suppressor T cells, killer T cells, killer cells, rod cells, cone cells, inner hair cells of the organ of Corti, outer hair cells of the organ of Corti, type I hair cells of the vestibular apparatus of the ear, type II cells of the vestibular apparatus of the ear, type II taste bud cells, olfactory neurons, basal cells of olfactory epithelium, type I carotid body cells, type II carotid body cells, Merkel cells, primary sensory neurons specialised for touch, primary sensory neurons specialised for temperature, primary neurons specialised for pain, proprioceptive primary sensory neurons, cholinergic neurons of the autonomic nervous system, adrenergic neurons of the autonomic nervous system, peptidergic neurons of the autonomic nervous system, inner pillar cells of the organ of Corti, outer pillar cells of the organ of Corti, inner phalangeal cells of the organ of Corti, outer phalangeal cells of the organ of Corti, border cells, Hensen cells, supporting cells of the vestibular apparatus, supporting cells of the taste bud, supporting cells of the olfactory epithelium, Schwann cells, satellite cells, enteric glial cells, neurons of the central nervous system, astrocytes of the central nervous system, oligodendrocytes of the central nervous system, anterior lens epithelial cells, lens fibre cells, melanocytes, retinal pigmented epithelial cells, iris pigment epithelial cells, oogonium, oocytes, spermatocytes, spermatogonium, ovarian follicle cells, Sertoli cells, thymus epithelial cells and hepatocarcinoma cells or combinations thereof.

In a further embodiment of the first aspect, the reconstituted extracellular matrix is derived from cell lines derived from any of the cell types above.

According to a second aspect of the present invention, there is provided a method for synthesising a biomaterial scaffold, the method comprising:

a) isolating extracellular matrix from a target cell or tissue;

b) obtaining a particulate suspension of a);

c) forming polyelectrolyte complex fibers with the suspension of b) under interfacial polyelectrolyte complexation conditions; and

d) forming the scaffold from the fibers.

According to a third aspect of the present invention, there is provided a composite material comprising a polyelectrolyte complex and extracellular matrix.

In one embodiment of the third aspect, the extracellular matrix is obtained from a cell or tissue type as described above or a combination thereof. In another embodiment of the third aspect, the composite material comprises a constituent element of a biomaterial scaffold.

According to a fourth aspect of the present invention, there is provided a biomaterial scaffold comprising reconstituted extracellular matrix, polyelectrolyte complex fibers and seeded cells, wherein the extracellular matrix is derived from the same or similar cell type as the seeded cells.

According to a fifth aspect of the present invention, there is provided a biomaterial scaffold comprising reconstituted extracellular matrix, polyelectrolyte complex fibers and seeded cells, wherein the extracellular matrix is derived from the same cell type as the seeded cells.

According to a sixth aspect of the present invention, there is provided a method for proliferating, differentiating or maintaining the differentiated phenotype and functions of seeded cells, the method comprising seeding a desired cell type or cell types on a biomaterial scaffold as described above and culturing said seeded cells under conditions conducive to proliferation, differentiation or maintaining the differentiated phenotype and functions of the seeded cells.

The summary of the invention described above is not limiting and other features and advantages of the invention will be apparent from the following detailed description of the preferred embodiments, as well as from the claims.

In the context of this specification, the term “comprising” means “including principally, but not necessarily solely”. Furthermore, variations of the word “comprising”, such as “comprise” and “comprises”, have correspondingly varied meanings.

### BRIEF DESCRIPTION OF THE DRAWINGS

FIG. 1: UV spectrophotometry of supernatants, before and after treatment with DNAse. Treatment with BSA, at the same concentration as DNAse, was used as the control.

FIG. 2: Mass of nucleic acid extracted into 200 μL of Solution B (10 mM magnesium chloride, 1 mM calcium chloride, 1 mM PMSF) containing different quantities of DNAse.

FIG. 3: Immunohistochemistry of fibers, demonstrating the presence of (a) fibronectin; (b) collagen; (c) heparan sulfate proteoglycans. Ab: Antibody, ECM: extra-cellular matrix.

FIG. 4: MC-3T3 cells grown on (a) ECM scaffold, and (b) Control scaffold.

FIG. 5: Supernatant albumin concentrations in primary hepatocyte culture vs. time.

FIG. 6: Fluorescent micrograph of HepG2 cells stably transduced with Green Fluorescent Protein (GFP) cultured on ECM Scaffold comprising reconstituted extracellular matrix from rat liver tissue, 24 hours after seeding.

### DETAILED DESCRIPTION OF THE PREFERRED EMBODIMENTS

The preferred embodiments of the invention will now be described in more detail, including, by way of illustration only, with respect to the examples that follow.

### Preparation of Polyelectrolyte Complex

The polyectrolyte complex forming the basis of a scaffold includes a polyanion and a polycation, which are collectively referred to as polyelectrolytes or polyions. The complex preferably includes a cross-linker. The cross-linker can crosslink the polyelectrolytes within a strand of fiber thus inhibiting secondary complexation of polyelectrolytes between adjacent fibers during the entanglement treatment. The fibers used may be prepared in any suitable manner, such as by interfacial polyelectrolyte complexation. The fibers are entangled in order to create the scaffold. The scaffold is then seeded with a target cell type for growth of that cell upon the scaffold. The target cells growing on the scaffold may be referred to as “seeded cells”.

The fibers may be entangled with a suitable fluid such as water. For example, the fibers may be entangled by hydroentanglement, also conventionally referred to as spunlace, jet entanglement, water entanglement, hydraulic needling, or hydrodynamic needling. A technique for preparing fibers comprising a cross-linker and entangling those fibers using a hydroentanglement technique is described in PCT Application PCT/SG2005/000198 “Scaffold and Method of Forming Scaffold by Entangling Fibres” by the present inventors, the contents of which are incorporated herein by reference.

Hydroentanglement techniques conventionally used in the textile industry for consolidating nonwoven webs of fibers may be suitable in some applications. Some suitable conventional hydroentanglement processes are described in U. Munstermann et al. “Hydroentanglement process”, in Nonwoven Fabrics Raw Materials, Manufacture, Applications, Characteristics, Testing processes, edited by W. Albrecht, H. Fuchs, W. Kittelmann, Wiley-VCH: Weinheim, 2000; and U.S. Pat. No. 6,112,385 to Gerold Fleeissner and Alfred Watzl, issued Sep. 5, 2000, the contents of each of which are incorporated herein by reference.

### Polyelectrolyte Fibers

The fibers used in the present invention may have any suitable size and shape. The Average diameters of the fibers may be in the range of tens of microns such as about 1-100 microns, about 10-100 microns, about 15 to 85 microns, about 30 to 70 microns. The lower limit of the diameter may be dictated by the mechanical properties of the fibers. The upper limit of the diameter may depend on how the particular fiber material can be effectively entangled by hydroentanglement. The lengths of fibers may also vary, depending on the application. For example, the lengths may be in the range of 1 to 1,000 mm, such as about 50 to 900 mm, about 150 to 800 mm, about 300 to 750 mm, 400 to 600 mm. The fibers may be pre-treated, such as washed, before being entangled. As can be appreciated, wetted fibers can be easier to manipulate than dry fibers.

Fibers can include any polyelectrolyte complex. A polyelectrolyte complex can be formed by two oppositely charged polyelectrolyte molecules, a polyanion and a polycation. A polyelectrolyte is typically a macromolecular species that upon being placed in water or any (other ionizing solvent dissociates into a highly charged polymeric molecule. Exemplary polyelectrolyte complexes include alginate-chitosan, heparin-chitosan, chondroitin sulfate-chitin, hyaluronic acid-chitosan, DNA-chitin, RNA-chitin, poly(glutamic acid)-poly(ornithic acid), polyacrylic acid-poly(lysine), and poly(ethyleneimine)-gellan complexes, and the like.

Suitable polyelectrolyte materials for forming polyelectrolyte complexes include natural polyelectrolytes, synthetic polyelectrolytes, chemically modified biopolymers and the like. Exemplary polyelectrolyte materials include carboxylated polymers; aminated polymers such as poly(ethyleneimine); chitin and chitosan and their derivatives; acrylate polymers; nucleic acids such as DNA and RNA; histone proteins; acidic polysaccharides and their derivatives such as chondroitin sulfate, heparin and alginate; poly(amino acids) such as poly(lysine) and poly(glutamic acid); hyaluronic acid; poly(ornithic acid); polyacrylic acid; gellan; and the like. The choice of the polyelectrolyte materials may depend on the application in which the scaffold is to be used and the particular processes employed for forming the fibers. For example, the alginate and chitosan pair may be used in biomedical applications because they have desirable physical, chemical and biochemical properties.

Polyelectrolyte complexes can form when oppositely charged polyelectrolytes are brought close to each other in a process known as interfacial polyelectrolyte complexation. For example, alginate (a polyanion) and chitosan (a polycation) can form a polyelectrolyte complex in such a process. In such a process, a polyanion solution and a polycation solution are brought close to each other, forming an interface. In the interface region, local complexation can occur. Complexation refers to the binding of two oppositely charged polyelectrolytes to form a polyelectrolyte complex. The polyelectrolyte complex formed can become insoluble due to neutralization of charges. Thus, a strand of fiber can be drawn from the interface region and polyelectrolyte complex fibers can be prepared.

The complexation process of forming polyelectrolyte complexes in each fiber is referred to herein as “primary” polyelectrolyte complexation. The polyelectrolyte complexes between adjacent fibers may also form larger complexes through “secondary” polyelectrolyte complexation, particularly when water is introduced into the fibers.

When fibers are pressed against each other in water, secondary polyelectrolyte complexation can occur due to the attraction between the oppositely charged groups from the adjacent fibers. As a result of the secondary polyelectrolyte complexation, a larger polyelectrolyte complex is formed, which holds fibers together. The fibers contain a polyelectrolyte complex (also called polyion complex) and a cross-linker. The cross-linker can crosslink the polyelectrolytes within a strand of fiber thus inhibiting secondary complexation of polyelectrolytes between adjacent fibers during the entanglement treatment. Secondary complexation of polyelectrolytes is considered inhibited if it is prevented or reduced. The cross-linker can include silicon, which can bind to the polyelectrolytes through Si—O bonds. For example, the cross-linker can include siloxane bonds (Si—O—Si), such as in silica.

The relative amount of the cross-linker in the fibers can be readily determined by persons skilled in the art, depending on the application and the polyelectrolytes used. When the fibers are formed by interfacial polyelectrolyte complexation with alginate and chitosan as the polyelectrolytes and TEOS as the precursor for the cross-linker, the weight ratio of chitosan, alginate and TEOS in the interfacial region can be between about 8:1:0 and about 1:16:19. It may be advantageous if the ratio is from about 8:1:3.7 to about 1:16:9.4.

As can be appreciated, other silica precursors may be used. For example, TEOS may be replaced by or used with tetramethyl orthosilicate (TMOS), Si(OCH₃)₄ or by TPOS, aminopropyltriethoxysilane (APTS).

Fibers may be formed with any suitable interfacial polyelectrolyte complexation technique, including conventionally known techniques such as wet spinning techniques, with possible modifications to incorporate the cross-linker and the modifier. The conventional fiber formation techniques are understood and can be readily performed by persons skilled in the art and will not be described in detail herein. Further details of forming fibers by interfacial polyelectrolyte complexation can be found in, for example, Andrew C A. Wan et al., “Encapsulation of biologies in self-assembled fibers as biostructural units for tissue engineering”, Journal of Biomedical Materials Research, (2004), vol. 71A, pp. 586-595 (“Wan I”), Andrew C A. Wan et al., “Mechanism of Fiber Formation by Interfacial Polyelectrolyte Complexation”, Macromolecules, (2004), vol. 37, pp. 7019-7025 (“Wan II”); Masato Amaike et al., “Sphere, honeycomb, regularly spaced droplet and fiber structures of polyion complexes of chitosan and gellan,” Macromolecules Rapid Communication, (1998), vol. 19, pp. 287-289; U.S. patent application publication number 2003/0055211 to George A. F. Roberts, published Mar. 20, 2003; and U.S. Pat. No. 5,836,970 to Abhay S. Pandit, issued Nov. 17, 1998; the contents of each of which are incorporated herein by reference.

### Extracellular Matrix

Extracellular matrices (ECM) that are derived from animal tissue are a rich source of bioactive ligands and growth factors, and have been used as scaffolds for tissue engineering². As these scaffolds are tissue-derived, their size, shape and configuration are limited by the dimensions and form of the original tissue. One potential source of ECM are cells that are grown in culture. These may include tumorized cell-lines or passaged primary cells. A second alternative would be to isolate the ECM from animal tissue and subsequently reconstitute it into the desired scaffold geometry and dimensions.

In the present invention, ECM is isolated from cells grown in culture or derived from tissue, and reconstructed into fibrous scaffolds based on polyelectrolyte complexes. Focusing on ECM from MC-3T3, an osteoblast cell-line, HepG2, a hepatocarcinoma cell line and rat liver, the presentation of ECM components such as fibronectin, collagen and heparan sulfate proteoglycan on these scaffolds is demonstrated by immunohistochemistry. Retention of the native characteristics of the ECM is shown by culturing MC-3T3 cells on their reconstituted ECM. The potential applicability of the ECM scaffolds was demonstrated by the ability of the reconstituted HepG2 ECM scaffolds to support the growth and function of primary rat hepatocytes.

The present invention is not however limited to the HepG2 and MC-3T3 cells or rat liver. Any cell or tissue type may be used in the present invention as a source of ECM which can be reconstructed into the fibrous scaffold described above. Examples of such cells include but are not limited by the following: embryonic stem cells, adult stem cells, blast cells, cloned cells, placental cells, keratinocytes, basal epidermal cells, urinary epithelial cells, salivary gland cells, mucous cells, serous cells, von Ebner's gland cells, mammary gland cells, lacrimal gland cells, ceruminous gland cells, eccrine sweat gland cells, apocrine sweat gland cells, Moll gland cells, sebaceous gland cells, Bowman's gland cells, Brunner's gland cells, seminal vesicle cells, prostate gland cells, bulbourethral gland cells, Bartholin's gland cells, Littre gland cells, uterine endometrial cells, goblet cells of the respiratory or digestive tracts, mucous cells of the stomach, zymogenic cells of the gastric gland, oxyntic cells of the gastric gland, insulin-producing β cells, glucagon-producing a cells, somatostatin-producing DELTA cells, pancreatic polypeptide-producing cells, pancreatic ductal cells, Paneth cells of the small intestine, type II pneumocytes of the lung, Clara cells of the lung, anterior pituitary cells, intermediate pituitary cells, posterior pituitary cells, hormone secreting cells of the gut or respiratory tract, thyroid gland cells, parathyroid gland cells, adrenal gland cells, gonad cells, juxtaglomerular cells of the kidney, macula densa cells of the kidney, peri polar cells of the kidney, mesangial cells of the kidney, brush border cells of the intestine, striated ducted cells of exocrine glands, gall bladder epithelial cells, brush border cells of the proximal tubule of the kidney, distal tubule cells of the kidney, conciliated cells of the ductulus efferens, epididymal principal cells, epididymal basal cells, hepatocytes, fat cells, type I pneumocytes, pancreatic duct cells, nonstriated duct cells of the sweat gland, nonstriated duct cells of the salivary gland, nonstriated duct cells of the mammary gland, parietal cells of the kidney glomerulus, podocytes of the kidney glomerulus, cells of the thin segment of the loop of Henle, collecting duct cells, duct cells of the seminal vesicle, duct cells of the prostate gland, vascular endothelial cells, synovial cells, serosal cells, squamous cells lining the perilymphatic space of the ear, cells lining the endolymphatic space of the ear, choroid plexus cells, squamous cells of the pia-arachnoid, ciliary epithelial cells of the eye, corneal endothelial cells, ciliated cells having propulsive function, ameloblasts, planum semilunatum cells of the vestibular apparatus of the ear, interdental cells of the organ of Corti, fibroblasts, pericytes of blood capillaries, nucleus pulposus cells of the intervertebral disc, cementoblasts, cementocytes, odontoblasts, odontocytes, chondrocytes, osteoblasts, osteocytes, osteoprogenitor cells, hyalocytes of the vitreous body of the eye, stellate cells of the perilymphatic space of the ear, skeletal muscle cells, heart muscle cells, smooth muscle cells, myoepithelial cells, red blood cells, platelets, megakaryocytes, monocytes, connective tissue macrophages, Langerhan's cells, osteoclasts, dendritic cells, microglial cells, neutrophils, eosinophils, basophils, mast cells, plasma cells, helper T cells, suppressor T cells, killer T cells, killer cells, rod cells, cone cells, inner hair cells of the organ of Corti, outer hair cells of the organ of Corti, type I hair cells of the vestibular apparatus of the ear, type II cells of the vestibular apparatus of the ear, type II taste bud cells, olfactory neurons, basal cells of olfactory epithelium, type I carotid body cells, type II carotid body cells, Merkel cells, primary sensory neurons specialised for touch, primary sensory neurons specialised for temperature, primary neurons specialised for pain, proprioceptive primary sensory neurons, cholinergic neurons of the autonomic nervous system, adrenergic neurons of the autonomic nervous system, peptidergic neurons of the autonomic nervous system, inner pillar cells of the organ of Corti, outer pillar cells of the organ of Corti, inner phalangeal cells of the organ of Corti, outer phalangeal cells of the organ of Corti, border cells, Hensen cells, supporting cells of the vestibular apparatus, supporting cells of the taste bud, supporting cells of the olfactory epithelium, Schwann cells, satellite cells, enteric glial cells, neurons of the central nervous system, astrocytes of the central nervous system, oligodendrocytes of the central nervous system, anterior lens epithelial cells, lens fibre cells, melanocytes, retinal pigmented epithelial cells, iris pigment epithelial cells, oogonium; oocytes, spermatocytes, spermatogonium, ovarian follicle cells, Sertoli cells, and thymus epithelial cells, or combinations thereof and cell lines derived thereof.

The animal tissue may be obtained from any animal tissue but is particularly selected from the group comprising skin, liver, pancreas, kidney, bone marrow, muscle, heart, lungs, gastrointestinal tract, brain and small intestinal submucosa.

The material may be treated with an appropriate enzyme, for example, to assist in the removal of undesirable components. Appropriate enzymes include for example, DNAse I. The concentration of the DNAse I may be about 0.005-1%, about 0.008-0.8%, about 0:011-0.5%, about 0.014-0.2%. More typically the concentration of the Dnase I may be about 0.016-0.08%, about 0.018-0.05%, about 0.019-0.03%.

The present invention is illustrated by reference to the examples herein. The invention is not, however limited to the specific exemplified embodiments. For example, in the preparation of the scaffolds, when chitosan is used, it may be used in a suitable acid, such as acetic acid at any appropriate volume fraction. To illustrate, the chitosan solution may be in the range of about 0.1-5%, typically about 0.2-4%, about 0.2-3%, about 0.3-2%. More typically the chitosan solution may be in the range of about 0.4-1%, about 0.45-0.75%. The acetic acid may be in the range of about 0.01-5%, typically about 0.5-2%, about 0.8-1.1%, about 0.1-4%.

As described herein, the ECM is incorporated into the polyelectrolyte complex fibers, preferably after being dispersed to a particulate form. Any suitable means of dispersion to a particulate form may be utilized. In the exemplified embodiments, the ECM is dispersed to a particular solution in 1% alginate. It is by such dispersal in the solution that the ECM becomes functionally associated with the fibers.

Any suitable solution, such as an alginate solution may be used. To illustrate, an alginate solution may be used in the range of about 0.1-5%, typically about 0.3-4%, about 0.5-3%, about 0.6-2%. More typically the alginate solution may be in the range of about 0.7-1.5%, about 0.9-1.1%.

As described herein, the hydrogel scaffolds incorporated with ECM may be formed through methods of the invention. In the illustrated embodiment, the hydrogel formation includes use of the heterobiofunctional PEG, NHS-PEG-MAL (Nektar). Together with the description provided herein, the skilled addressee will appreciate that any suitable agent may be used. To illustrate, where the method utilizes NHS-PEG-MAL, the volume of NHS-PEG-MAL (aq) (Nektar) may be in the range of about 1-10 mg/mL, typically about 2-9 mg/mL, about 3-8 mg/mL, about 4-7 mg/mL. More typically the volume of NHS-PEG-MA (aq) (Nektar) may be in the range of about 5-6 mg/mL.

In the preparation of a hydrogel type polyelectrolyte complex fiber scaffold, the scaffold may be air-dried and treated with deionized water to bring about swelling of the fibers and hydrogel scaffold formation. In the illustrated embodiment, the weight of fibers was 1-2 mg. It will be appreciated that any appropriate amount may be used. For example, the weight of the air-dried collections of fibers may be in the range of about 0.1-10 mg, typically about 0.2-8 mg, about 0.5-6 mg, about 0.7-4. More typically the weight of the air-dried collections of fibers may be in the range of about 0.9-2 mg.

In the illustrated embodiment, the air dried collections of fibers are treated with deionized water (20-200 μL). It will be appreciated that any appropriate volume may be used, for example the volume may be in the range of about 1-1000 μL, about 3-900 μL, about 6-800 μL, about 9-600 μL, about 12-500 μL, 15-400 μL. More typically the volume of deionized water may be in the range of about 18-300 μL, about 19-250 μL.

As described herein, the ECM may be obtained from animal tissue. The animal tissue is typically cut into small pieces and is treated with a chelating agent preferably containing antibiotics. In the illustrated embodiment, the chelating agent is EDTA and the concentration of EDTA may be in the range of about 0.01-5%, about 0.02-0.08%, about 0.03-0.07%. More typically the concentration of EDTA may be in the range of about 0.04-0.06%.

This is typically followed by a buffer wash. In the illustrated embodiment, for cell lysis and extraction, the tissue is treated with a solution of 1% triton X-100 in 10 mM Tris buffer (pH 8), with the addition of a protease inhibitor cocktail and antibiotics, and shaken on an orbital shaker for 48 hr at 4° C. The concentration of triton X-100 may be in the range of about 0.01-10%, about 0.3-4%, about 0.5-3%. More typically the concentration of triton X-100 may be in the range of about 0.7-2%, about 0.9-1.3. The duration of shaking may be in the range of about 12-168 hours, about 15-140 hours, about 18-110 hours, about 22-90 hours, about 26-75 hours, about 30-70 hours. More typically, the duration of shaking may be in the range of about 35-60 hours, about 40-55 hours, about 45-50 hours.

In the illustrated example, the lysed tissue is rinsed for a further 48 hr at 4° C., changing the solution every 12 hr. The duration of rinsing may be in the range of about 12-168 hours, about 15-140 hours, about 18-110 hours, about 22-90 hours, about 26-75 hours, about 30-70 hours. More typically, the duration of rinsing may be in the range of about 35-60 hours, about 40-55 hours, about 45-50 hours.

In the illustrated example, the product is homogenized using a sonicator probe homogenizer at an amplitude of 61% until a particulate suspension is obtained. The amplitude may be in the range of about 1-100%, about 10-90%, about 20-80%, about 30-75%. More typically the amplitude may be in the range of about 40-70%, about 50-65%, about 58-63%.

In particular, advantageously, the ECM isolated from cells grown in culture or derived from tissue, and reconstructed into fibrous scaffolds based on polyelectrolyte complexes can be matched to the cells that are to be grown on that scaffold. That is to say, it is possible to use the same or similar cells in the extracellular matrix as the cells to be grown on the matrix.

Furthermore, the ECM can be derived from a cell type/tissue type chosen to provide differentiation signals to stem cells. For example, stem cells grown on a scaffold comprising reconstituted ECM from liver may be, able to differentiate into liver cells. ECM can also be derived from a cell line or tissue type chosen to provide a suitable environment to sustain the function of primary cells. In Example 6 hereafter, it is shown that primary hepatocytes from rat liver can maintain albumin secretion (a liver-specific function) for a longer period of time when cultured on a scaffold comprising reconstituted ECM from HepG2, a liver-like cell line compared to control chitosan-alginate scaffolds and hepatocytes grown-on tissue culture plates.

### Scaffold Application

The scaffolds prepared as described above have applications in many fields including tissue engineering, 3-D cell culturing, 3-D cell culture system for high-throughput drug screening, drug-releasing fabrics, containers for expansion of cells such as stem cells, and the like. More particularly the incorporation of extracellular matrix into the 3D matrices adapts the matrices for the investigation of the influence of the ECM on cell phenotype, and constitutes a promising approach to the engineering of functional tissue.

### EXAMPLES

The examples are intended to serve to illustrate this invention and should not be construed as limiting the general nature of the disclosure of the description throughout this specification.

### Example 1 Isolation of ECM

MC-3T3, an osteoblast cell line, and HepG2, a hepatocarcinoma cell line, were seeded at a density of 1.5×10⁴ cells/cm² and grown for 1 week with one change of medium in alpha MEM and DMEM (supplemented with 10% FBS, 1% P/S penicilin/streptomycin respectively.

To isolate the extracellular matrix (ECM), the medium was slowly aspirated from the tissue culture dish and washed twice with phosphate buffered saline. 1 mL of Solution A (1 mM Phenylmethanesulfonyl fluoride (PMSF, Fluka), 10 mM Tris(hydroxymethyl)aminomethanehydrochloride (TRIS) (Merck), pH8, 0.5% Sodium Deoxycholate) was applied to each 100 mm dish for 1 min. Following the removal of Solution A, each dish was washed with 1 mL of phosphate buffered saline. Then, 1 mL of deionized water was forcefully squirted onto the bottom of the petri dish to detach the ECM. The suspension was transferred into separate vials and centrifuged at 7500×g at 4° C. for 5 minutes. The supernatant was removed, after which 1 mL of Solution B (10 mM Magnesium chloride, 1 mM calcium chloride, 1 mM PMSF, 0.02% DNASE 1 from bovine pancreas (Sigma)) was added.

Next, the ECM was dispersed by vortexing and collected at the bottom of the vial. The vials were then placed on a Heidolph-Unimax shaker for 30 mins at an agitation rate of 250 rpm. The vials were centrifuged at 7500×g and 4° C. for 5 mins. The supernatant was removed and the ECM pellet was washed with deionized water by dispersion and centrifugation to remove residual DNAse. Alternatively, suspensions were consolidated and transferred to an Amicon Ultra Centrifugal Filter device (Millipore) and centrifuged at 1100×g at 4° C. (1 hr of centrifugation for every 1.5 ml of solution) The solid ECM was removed. 1% alginate was added and the suspension was drawn against an aqueous solution of 1.5% water-soluble chitin or 0.5% chitosan in 2% acetic acid for fiber formation.

For the DNAse study, MC-3T3 cells were cultured in 24-well plates and the reagents were scaled down as follows: Solution A, 200 μL; phosphate buffered saline, 300 μL; deionized water, 200 μL; Solution B, 200 μL.

### Example 2 Characterization of ECM

Immunohistochemistry of the ECM components was performed by using antibodies against fibronectin and collagen Type I (Acris Antibodies, GmbH). The primary antibodies were rabbit polyclonal antibody to fibronectin and collagen Type I whereas the secondary antibody was FITC labeled F(ab′)2 fragment of affinity purified anti-Rabbit IgG (Acris Antibodies, GmbH). Confocal microscopy was performed on an Olympus Fluoview 300 confocal unit with a 488 nm laser. Green fluorescence was observed using a 510 nm long pass and a 530 nm short pass filter.

### Example 3 Preparation of HepG2 ECM Reconstituted Scaffolds

To prepare the polycation precursor, tetraethylorthosilicate (TEOS) was first hydrolyzed by mixing TEOS and 0.15 M acetic acid at a ratio of 1:9, that is to say, 0-25% by volume of TEOS and vortexing until a homogenous solution was obtained. Hydrolyzed TEOS was then added to a 0.5% chitosan solution in 1% acetic acid at a volume fraction of 25%. To prepare the polyanion precursor, HepG2 ECM was dispersed in a 1% alginate solution in deionized water by tituration and vortexing.

For incorporation of ECM into the polyelectrolyte complex fibers, the original film-like material had to be first dispersed to a particulate form as discussed above. This could be achieved by simply titurating the isolated ECM With deionized water, transferring the suspension to fresh vials followed by centrifugation to obtain the ECM pellet. The ECM could then be dispersed to a particulate suspension in 1% alginate. For storage of ECM, the stability of the suspension appeared to be better in deionized water as compared to alginate. As such, the ECM was stored in deionized water prior to use.

30 μL of the polycation and 20 μL of the polyanion precursors were placed in 3 mm PTFE channels, close to but not touching each other. A pair of forceps was used to bring the droplets in contact and an upward motion was applied to form fiber. The nascent fiber was adhered onto the rotating arms of a roll-up apparatus and fiber was drawn continuously until the polyelectrolyte solutions were depleted and/or fiber termination occurred. The dry fibers obtained from the roll-up apparatus were transferred to 1.7-mL microcentrifuge tubes and weighed. Approximately 1.5 mL of deionized water was then added to wash the fibers for 5 min. The washed fibers were then transferred onto a frit in a die and a stream of deionized water was passed through the die at a flow rate of 300-350 mL/min for 1 min to entangle the fibers. The water flow rate was then reduced to 5-35 mL/min, and the fibers were washed for another 5 min. The formed scaffolds were subsequently transferred to a 96-well plate containing 70% ethanol prior to use.

### Example 4 Primary Hepatocyte Culture

Hepatocytes were harvested from Wistar rats by a two-step, in situ collagenase perfusion procedure, as previously reported.³ The cells were dispersed and cultured in a chemically defined medium, Gibco™ HepatoZYME-SFM supplemented with 10% fetal bovine serum. Cells were seeded on the scaffolds in 96-well plates at a density of 1-2×10⁵ cells per well. Cell culture supernatants were sampled daily and replaced with an equal volume of fresh media. The samples were frozen at −20° C. prior to the assay, at which time they were thawed and centrifuged at 7500×g for 4 min, in order to pellet and remove any entrapped cells. The concentrations of albumin in the samples were measured by ELISA (R&D Systems), according to manufacturer's instructions.

### Example 5 Discussion

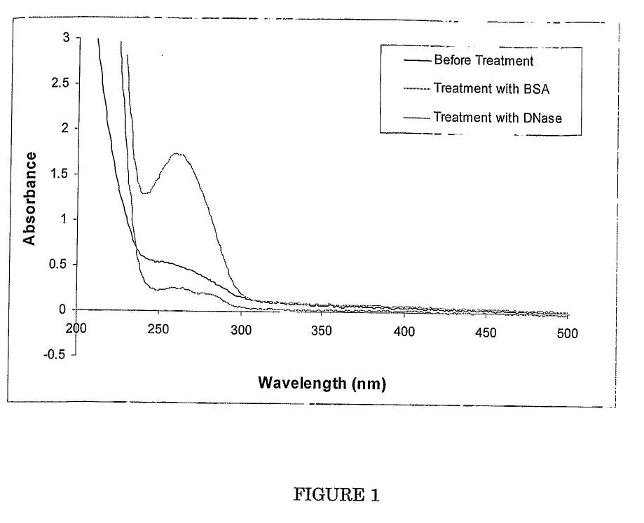

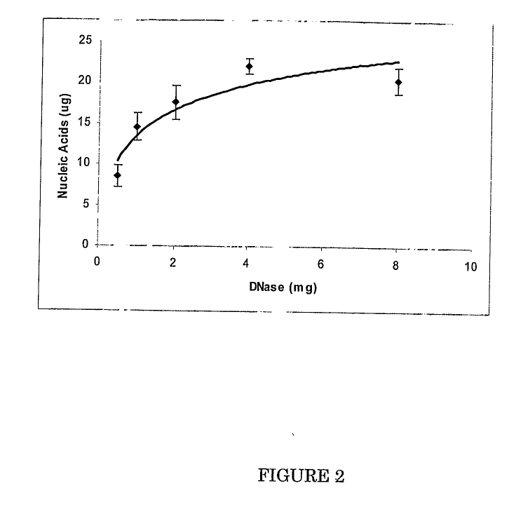

The procedure for extracellular matrix isolation was optimized for the isolation of extracellular matrix from MC-3T3, a mouse osteoblast cell line and HepG2, a hepatocellular carcinoma cell line. Modifications were made with regard to the duration of exposure to the deoxycholate solution and the latter solution volume as these affected the removal of the cellular fraction. Over-exposure resulted in poor yield, whereas under-exposure resulted in cellular residue in the isolated material. As an additional step, we introduced DNAse to remove nucleic acids from the extracellular matrix. (FIG. 1) UV spectrophotometry of the collected supernatants demonstrated the effectiveness of the protocol. FIG. 2 establishes the optimal quantity of DNAse for our protocol.

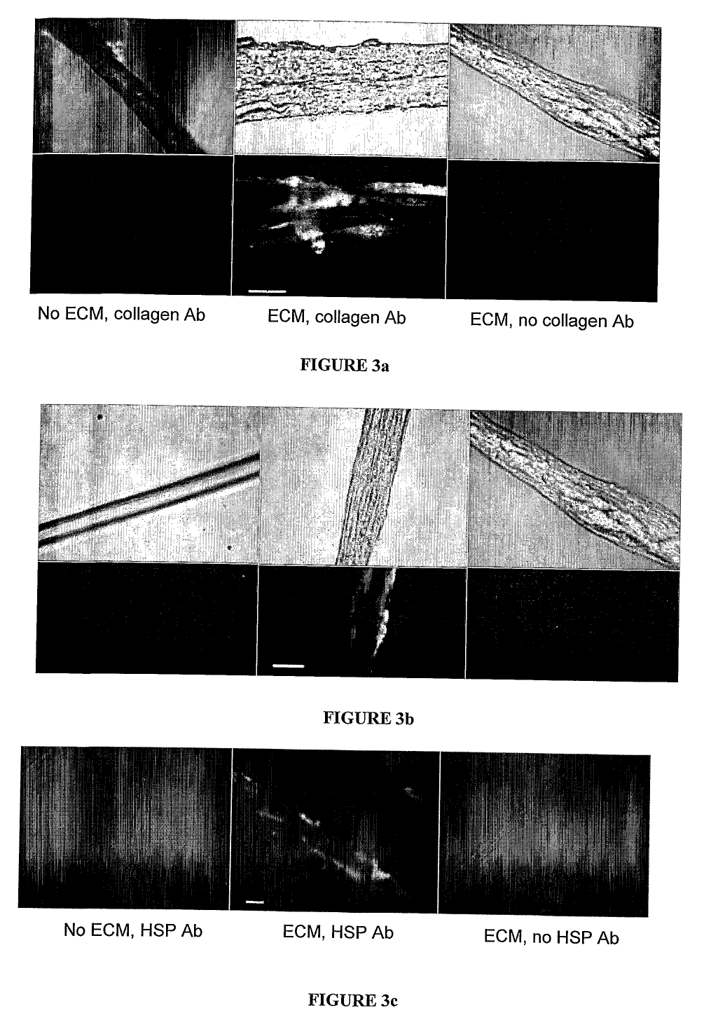

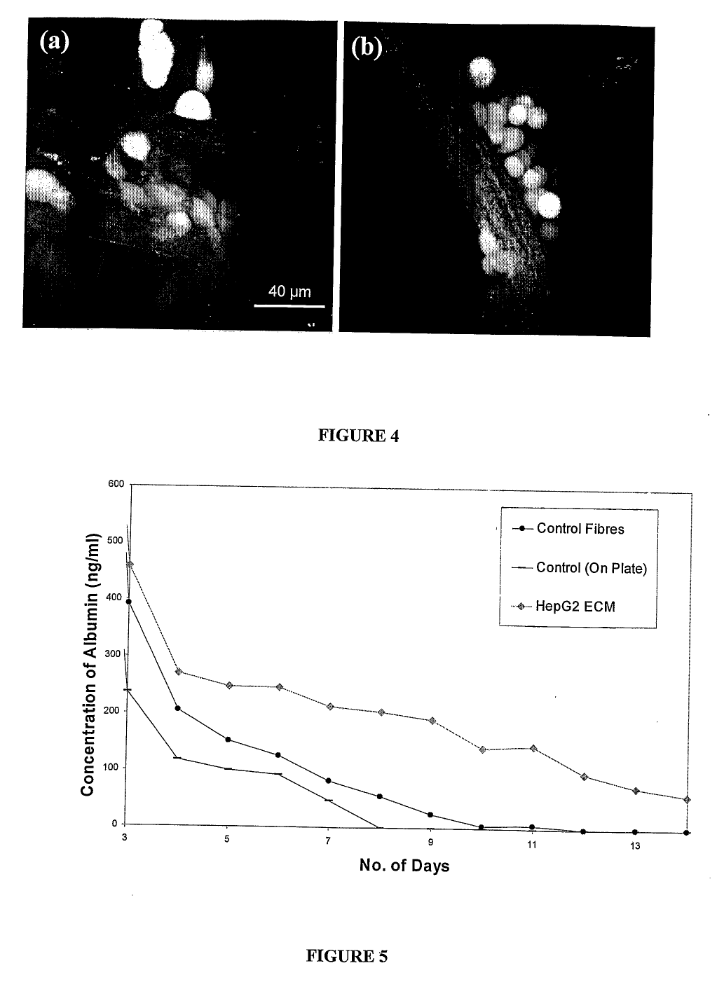

Immunofluorescence of the reconstituted ECM scaffold was performed using antibodies against fibronectin, collagen and heparan sulfate proteoglycan, these being the major components of both osteoblast and liver ECM.⁵ These three ECM components were shown to be present, as illustrated for the case of the reconstituted MC3T3 ECM scaffold FIG. 3).

The fibrous scaffolds (containing reconstituted ECM) were fabricated by interfacial polyelectrolyte complexation, as described previously.⁶ ECM was dispersed in alginate and drawn up into fiber by forming a complex with either water-soluble chitin or chitosan.

FIG. 2 shows the confocal micrographs of MC-3T3 cells grown on scaffolds of reconstituted MC-3T3 ECM, compared to those grown on scaffolds without the ECM. Cells growing on the ECM scaffolds were able to spread out on the fibers, while cells growing on the non-ECM scaffolds were spherical and clustered. Cell adhesion on these scaffolds were likely to be mediated by the ECM molecules, collagen and fibronectin, which both contain the RGD sequence motif that binds to the integrin receptor on a wide variety of cell types.

The attractiveness of being able to reconstitute ECM from a wide variety of cell lines lies in the potentially limitless selection of ECM for 3D cell culture. Current models in cell biology and strategies in tissue engineering employ proteins whose functions and usefulness have been well established e.g. collagen, fibronectin and fibrin. Matrigel, a solubilized basement membrane preparation derived from Engelbreth-Holm-Swarn sarcoma is also a popular choice. In all probability, the ideal ECM for 3D tissue culture of a particular cell type would be the ECM native to the cells in question. For example, to recreate the stem cell niche in the bone marrow, one would use the ECM secreted by a bone narrow cell line (such as a mesenchymal stem cell line), whose ECM composition would be expected to be close to that of bone marrow ECM.

### Example 6 Effect of ECM-Reconstituted Scaffolds on Cell Growth

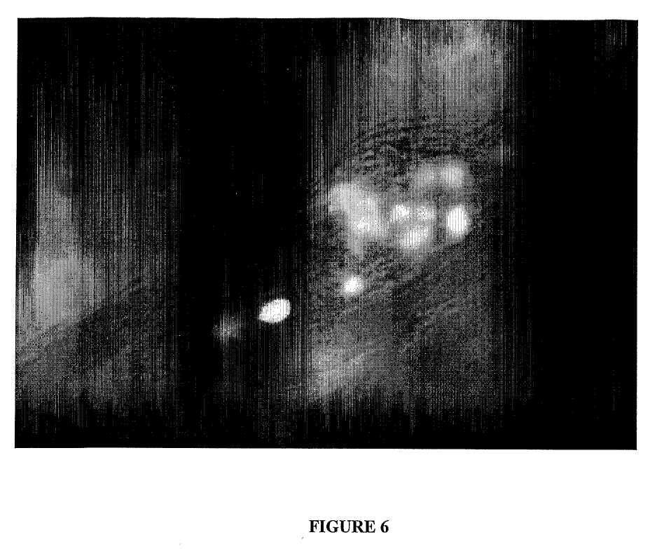

Primary hepatocytes isolated from collagenase-perfused rat liver were cultured on scaffolds incorporating ECM from HepG2, a hepatocellular carcinoma cell line. The ECM scaffold was compared with control chitosan-alginate scaffolds and hepatocytes grown on tissue culture plates. Albumin synthesis by the cells was used as a measure of hepatocyte function. FIG. 5 shows the concentration of albumin in the culture supernatant, measured by ELISA, over a two week period. The results demonstrate the positive influence of the ECM-reconstituted scaffolds in maintaining hepatocyte viability and function for up to two weeks in culture. The observation may be partially attributed to provision of cell-adhesive sites on the ECM molecules to the hepatocytes.

Other experiments conducted by the inventors have shown that the different proteins present in liver ECM vary in their ability to support hepatocyte function. For example, cells grown on Type I collagen scaffolds fare a lot better than those cultured on laminin scaffolds, a finding which is consistent with recently published data.⁷ This observation reinforces the advantage of using ‘whole’ ECM, rather than isolated ECM components, as the exact interplay of the different components and factors in the natural environment is unknown.

### Example 7 Impregnation of a Hydrogel Type Polyelectrolyte Complex Fiber Scaffold with ECM

5.5 mg/mL of NHS-PEG-MAL (aq) (Nektar) was vortexed with equal volume of 2% chitosan (aq) (NOF Corporation) for half hour. The resulting chitosan-PEG-MAL conjugate mixture was combined with alginic acid under conditions of interfacial polyelectrolyte complexation to form fiber, as in Example 3. When air-dried collections of these fibers (1-2 mg) were treated with deionized water (20-200 μL), extensive swelling of the fibers occurred, resulting in the immediate formation of a hydrogel scaffold.

In the above procedure, ECM could be dispersed into the alginic acid solution by tituration and vortexing, prior to incorporation into fiber. In this way, the hydrogel scaffold could be incorporated with ECM.

### Example 8 Isolation of ECM from Rat Liver Tissue

ECM could be isolated from liver and homogenized by sonication into a particulate form. Rat liver was cut into small pieces under sterile conditions and washed in a solution of 0.05% EDTA in 10 mM TRIS buffer, containing antibiotics (100 U/ml penicilin, 100 ug/ml streptomycin and 0.025 ug/ml amphotericin B). This was followed by a buffer wash. For cell lysis and extraction, the tissue was treated with a solution of 1% triton X-100 in 10 mM Tris buffer (pH 8), with the addition of a protease inhibitor cocktail and antibiotics, and shaken on an orbital shaker for 48 hr at 4° C. The lysed tissue was subsequently rinsed with 10 mM Tris (pH=8.0) containing the antibiotics and protease inhibitor cocktail for a further 48 hr at 4° C., change the solution every 12 hr. Nucleic acid digestion was carried out using a solution of 0.02% Dnase I in 10 mM Tris (pH=8.0) at 37° C., overnight. In addition to the same antibiotics and EDTA free protease inhibitor cocktail, the latter solution also contained 10 mM MgCl+1 mM CaCl₂. The product was rinsed as before, then homogenized using a sonicator probe homogenizer at an amplitude of 61% until a particulate suspension was obtained. The resulting tissue-derived ECM particulates were reconstituted into polyelectrolyte complex fibrous scaffolds as described for cell-line derived ECM in Example 3. Immunofluorescence labeling demonstrated the presence of heparan sulfate-proteoglycan on the fibers. HepG2 cells stably transduced with Green Fluorescent Protein (GFP) exhibited good adhesion onto these fibrous scaffolds (FIG. 6).

Any description of prior art documents herein, or statements herein derived from or based on those documents, is not an admission that the documents or derived statements are part of the common general knowledge of the relevant art in Australia or elsewhere.

While the invention has been described in the manner and detail as above, it will be appreciated by persons skilled in the art that numerous variations and/or modifications including various omissions, substitutions, and/or changes in form or detail may be made to the invention as shown in the specific embodiments without departing from the spirit or scope of the invention as broadly described. The present embodiments are, therefore, to be considered in all respects as illustrative and not restrictive.

### REFERENCES

1. P. X. Ma, Scaffolds for tissue fabrication, Materials Today, May 2004, p. 30-40.
2. Badylak, S, Xenogeneic extracellular matrix as a scaffold for tissue reconstruction, Transplant Immunology 12 (2004) 367-377.
3. Chia S. M., Leong K. W., Li. J., XU X., Zeng K., Er P. N., Gao S., Yu H.: Hepatocyte encapsulation for enhanced cellular functions, Tissue Engineering, 6(5) (2000), 481-95.
4. Hedman K., Markku K., Alitalo K., Vaheri A., Johansson S., Hook M. Isolation of the pericellular matrix of human fibroblast cultures, J. Cell Biology, 81 (1979), 83-91.
5. El-Amin S. F., Lu H. H., Khan Y., Burems J., Mitchell J., Tuan R. S., Laurencin C. T. Extracellular matrix production by human osteoblasts cultured on biodegradable polymers applicable for tissue engineering, Biomaterials, 24(7) (2003), 1213-21.
6. Wan A. C. A., Tai B. C. U., Leck K. J., Ying J. Y. Silica-incorporated polyelectrolyte-complex fibers as tissue engineering scaffolds, Advanced Materials, 18 (2006) 641-44.
7. Flaim C. J., Chien S., Bhatia S. N. An extracellular matrix microarray for probing cellular differentiation, Nature Methods, 2(2) (2004), 119-25.

1. A biomaterial scaffold comprising:
a) reconstituted extracellular matrix; and
b) polyelectrolyte complex fibers;
wherein the matrix and the fibers are functionally associated.

2. The biomaterial scaffold of claim 1, wherein the polyelectrolyte complex fibers are comprised of a polycation precursor and a polyanion precursor.

3. The biomaterial scaffold of claim 2, wherein the polycation precursor is sodium alginate.

4. The biomaterial scaffold of claim 2 wherein reconstituted extracellular matrix is incorporated into the polycation precursor and the polyanion precursor.

5. The biomaterial scaffold of claim 2 wherein reconstituted extracellular matrix is incorporated into the polycation precursor or the polyanion precursor.

6. The biomaterial scaffold of claim 2 wherein reconstituted extracellular matrix is incorporated into the polyanion precursor.

7. The biomaterial scaffold of claim 1, wherein the reconstituted extracellular matrix is derived from cultured cells or animal tissue.

8. The biomaterial scaffold of claim 7 wherein the animal tissue is selected from the group comprising skin, liver, pancreas, kidney, bone marrow, muscle, heart, lungs, gastro-intestinal tract, brain and small intestinal submucosa.

9. The biomaterial scaffold of claim 8, wherein the animal tissue is rat liver tissue.

10. The biomaterial scaffold of claim 1, wherein the reconstituted extracellular matrix is derived from cell culture or cells selected from any one of the group comprising embryonic stem cells, adult stem cells, blast cells, cloned cells, placental cells, keratinocytes, basal epidermal cells, urinary epithelial cells, salivary gland cells, mucous cells, serous cells, von Ebner's gland cells, mammary gland cells, lacrimal gland cells, ceruminpus gland cells, eccrine sweat gland cells, apocrine sweat gland cells, MpH gland cells, sebaceous gland cells, Bowman's gland cells, Brunner's gland cells, seminal vesicle cells, prostate gland cells, bulbourethral gland cells, Bartholin's gland cells, Littre gland cells, uterine endometrial cells, goblet cells of the respiratory or digestive tracts, mucous cells of the stomach, zymogenic cells of the gastric gland, oxyntic cells of the gastric gland, insulin-producing P cells, glucagon-producing a cells, somatostatin-producing DELTA cells, pancreatic polypeptide-producing cells, pancreatic ductal cells, Paneth cells of the small intestine, type II pneumocytes of the lung, Clara cells of the lung, anterior pituitary cells, intermediate pituitary cells, posterior pituitary cells, hormone secreting cells of the gut or respiratory tract, thyroid gland cells, parathyroid gland cells, adrenal gland cells, gonad cells, juxtaglomerular cells of the kidney, macula densa cells of the kidney, peri polar cells of the kidney, mesangial cells of the kidney, brush border cells of the intestine, striated ducted cells of exocrine glands, gall bladder epithelial cells, brush border cells of the proximal tubule of the kidney, distal tubule cells of the kidney, conciliated cells of the ductulus efferens, epididymal principal cells, epididymal basal cells, hepatocytes, fat cells, type I pneumocytes, pancreatic duct cells, nonstriated duct cells of the sweat gland, nonstriated duct cells of the salivary gland, nonstriated duct cells of the mammary gland, parietal cells of the kidney glomerulus, podocytes of the kidney glomerulus, cells of the thin segment of the loop of Henle, collecting duct cells, duct cells of the seminal vesicle, duct cells of the prostate gland, vascular endothelial cells, synovial cells, serosal cells, squamous cells lining the perilymphatic space of the ear, cells lining the endolymphatic space of the ear, choroid plexus cells, squamous cells of the pia-arachnoid, ciliary epithelial cells of the eye, corneal endothelial cells, ciliated cells having propulsive function, ameloblasts, planum semilunatum cells of (he vestibular apparatus of the ear, interdental cells of the organ of Corti, fibroblasts, pericytes of blood capillaries, nucleus pulposus cells of the intervertebral disc, cementoblasts, cementocytes, odontoblasts, odontocytes, chondrocytes, osteoblasts, osteocytes, osteoprogenitor cells, hyalocytes of the vitreous body of the eye, stellate cells of the perilymphatic space of the ear, skeletal muscle cells, heart muscle cells, smooth muscle cells, myoepithelial cells, red blood cells, platelets, megakaryocytes, monocytes, connective tissue macrophages, Langerhan's cells, osteoclasts, dendritic cells, microglial cells, neutrophils, eosinophils, basophils, mast cells, plasma cells, helper T cells, suppressor T cells, killer T cells, killer cells, rod cells, cone cells, inner hair cells of the organ of Corti, outer hair cells of the organ of Corti, type I hair, cells of the vestibular apparatus of the ear, type II cells of the vestibular apparatus of the ear, type II taste bud cells, olfactory neurons, basal cells of olfactory epithelium, type I carotid body cells, type II carotid body cells, Merkel cells, primary sensory neurons specialised for touch, primary sensory neurons specialised for temperature, primary neurons specialised for pain, proprioceptive primary sensory neurons, cholinergic neurons of the autonomic nervous system, adrenergic neurons of the autonomic nervous system, peptidergic neurons of the autonomic nervous system, inner pillar cells of the organ of Corti, outer pillar cells of the organ of Corti, inner phalangeal cells of the organ of Corti, outer phalangeal cells of the organ of Corti, border cells, Hensen cells, supporting cells of the vestibular apparatus, supporting cells of the taste bud, supporting cells of the olfactory epithelium, Schwann cells, satellite cells, enteric glial cells, neurons of the central nervous system, astrocytes of the central nervous system, oligodendrocytes of the central nervous system, anterior lens epithelial cells, lens fibre cells, melanocytes, retinal pigmented epithelial cells, iris pigment epithelial cells, oogonium, oocytes, spermatocytes, spermatogonium, ovarian follicle cells, Sertoli cells, and thymus epithelial cells, hepatocarcinoma or combinations thereof or cell lines derived therefrom.

11. The biomaterial scaffold of claim 1, wherein the reconstituted extracellular matrix is derived from an osteoblast cell line or a hepatocarcinoma cell line.

12. The biomaterial scaffold of claim 10, wherein the osteoblast cell line is MC-3T3.

13. The biomaterial scaffold of claim 10, wherein hepatocarcinoma cell line is HepG2.

14. The biomaterial scaffold of claim 1, further comprising at least one stabilising agent.

15. The biomaterial scaffold of claim 1, further comprising at least one biologically active agent, and wherein the biologically active agent comprises a plurality of cells seeded within the polyelectrolyte complex fibers.

16. The biomaterial scaffold according to claim 15, wherein the plurality of cells are selected from any one of the group comprising embryonic stem cells, adult stem cells, blast cells, cloned cells, placental cells, keratinocytes, basal epidermal cells, urinary epithelial cells, salivary gland cells, mucous cells, serous cells, von Ebner's gland cells, mammary gland cells, lacrimal gland cells, ceruminous gland cells, eccrine sweat gland cells, apocrine sweat gland cells, Moll gland cells, sebaceous gland cells, Bowman's gland cells, Brunner's gland cells, seminal vesicle cells, prostate gland cells, bulbourethral gland cells, Bartholin's gland cells, Littre gland cells, uterine endometrial cells, goblet cells of the respiratory or digestive tracts, mucous cells of the stomach, zymogenic cells of the gastric gland, oxyntic cells of the gastric gland, insulin-producing β cells, glucagon-producing a cells, somatostatin-producing DELTA cells, pancreatic polypeptide-producing cells, pancreatic ductal cells, Paneth cells of the small intestine, type II pneumocytes of the lung, Clara cells of the lung, anterior pituitary cells, 5 intermediate pituitary cells, posterior pituitary cells, hormone secreting cells of the gut or respiratory tract, thyroid gland cells, parathyroid gland cells, adrenal gland cells, gonad cells, juxtaglomerular cells of the kidney, macula densa cells of the kidney, peri polar cells of the kidney, mesangial cells of the kidney, brush border cells of the intestine, striated ducted cells of exocrine glands, gall bladder epithelial cells, brush border cells of the proximal tubule of the kidney, distal tubule cells of the kidney, conciliated cells of the ductulus efferens, epididymal principal cells, epididymal basal cells, hepatocytes, fat cells, type I pneumocytes, pancreatic duct cells, nonstriated duct cells of the sweat gland, nonstriated duct cells of the salivary gland, nonstriated duct cells of the mammary gland, parietal cells of the kidney glomerulus, podocytes of the kidney glomerulus, cells of the thin segment of the loop of Henle, collecting duct cells, duct cells of the seminal vesicle, duct cells of the prostate gland, vascular endothelial cells, synovial cells, serosal cells, squamous cells lining the perilymphatic space of the ear, cells lining the endolymphatic space of the ear, choroid plexus cells, squamous cells of the pia-arachnoid, ciliary epithelial cells of the eye, corneal endothelial cells, ciliated cells having propulsive function, ameloblasts, planum semilunatum cells of the vestibular apparatus of the ear, interdental cells of the organ of Corti, fibroblasts, pericytes of blood capillaries, nucleus pulposus cells of the intervertebral disc, cementoblasts, cementocytes, odontoblasts, odontocytes, chondrocytes, osteoblasts, osteocytes, osteoprogenitor cells, hyalocytes of the vitreous body of the eye, stellate cells of the perilymphatic space of the ear, skeletal muscle cells, heart muscle cells, smooth muscle cells, myoepithelial cells, red blood cells, megakaryocytes, monocytes, connective tissue macrophages, Langerhan's cells, osteoclasts, dendritic cells, microglial cells, neutrophils, eosinophils, basophils, mast cells, plasma cells, helper T cells, suppressor T cells, killer T cells, killer cells, rod cells, cone cells, inner hair cells of the organ of Corti, outer hair cells of the organ of Corti, type I hair cells of the vestibular apparatus of the ear, type II cells of the vestibular apparatus of the ear, type II taste bud cells, olfactory neurons, basal cells of olfactory epithelium, type I carotid body cells, type II carotid body cells, Merkel cells, primary sensory neurons specialised for touch, primary sensory neurons specialised for temperature, primary neurons specialised for pain, proprioceptive primary sensory neurons, cholinergic neurons of the autonomic nervous system, adrenergic neurons of the autonomic nervous system, peptidergic neurons of the autonomic nervous system, inner pillar cells of the organ of Corti, outer pillar cells of the organ of Corti, inner phalangeal cells of the organ of Corti, outer phalangeal cells of the organ of Corti, border cells, Hensen cells, supporting cells of the vestibular apparatus, supporting cells of the taste bud, supporting cells of the olfactory epithelium, Schwann cells, satellite cells, enteric glial cells, neurons of the central nervous system, astrocytes of the central nervous system, oligodendrocytes of the central nervous system, anterior lens epithelial cells, lens fibre cells, melanocytes, retinal pigmented epithelial cells, iris pigment epithelial cells, oogonium, oocytes, spermatocytes, spermatogonium, ovarian follicle cells, Sertoli cells, and thymus epithelial cells, hepatocarcinoma or combinations thereof or cell lines derived therefrom.

17. A method for synthesising a biomaterial scaffold, the method comprising:
a) isolating extracellular matrix from a target cell or tissue;
b) obtaining a particulate suspension of a);
c) forming polyelectrolyte complex fibers with the suspension of b) under interfacial polyelectrolyte complexation conditions; and
d) forming the scaffold from the fibers.

18. A composite material comprising a polyelectrolyte complex and extracellular matrix.

19. The composite material according to claim 18, wherein the extracellular matrix is obtained from cell culture or cells selected from any one of the group comprising embryonic stem cells, adult stem cells, blast cells, cloned cells, placental cells, keratinocytes, basal epidermal cells, urinary epithelial cells, salivary gland cells, mucous cells, serous cells, von Ebner's gland cells, mammary gland cells, lacrimal gland cells, ceruminpus gland cells, eccrine sweat gland cells, apocrine sweat gland cells, MpH gland cells, sebaceous gland cells, Bowman's gland cells, Brunner's gland cells, seminal vesicle cells, prostate gland cells, bulbourethral gland cells, Bartholin's gland cells, Littre gland cells, uterine endometrial cells, goblet cells of the respiratory or digestive tracts, mucous cells of the stomach, zymogenic cells of the gastric gland, oxyntic cells of the gastric gland, insulin-producing P cells, glucagon-producing α cells, somatostatin-producing DELTA cells, pancreatic polypeptide-producing cells, pancreatic ductal cells, Paneth cells of the small intestine, type II pneumocytes of the lung, Clara cells of the lung, anterior pituitary cells, intermediate pituitary cells, posterior pituitary cells, hormone secreting cells of the gut or respiratory tract, thyroid gland cells, parathyroid gland cells, adrenal gland cells, gonad cells, juxtaglomerular cells of the kidney, macula densa cells of the kidney, peri polar cells of the kidney, mesangial cells of the kidney, brush border cells of the intestine, striated ducted cells of exocrine glands, gall bladder epithelial cells, brush border cells of the proximal tubule of the kidney, distal tubule cells of the kidney, conciliated cells of the ductulus efferens, epididymal principal cells, epididymal basal cells, hepatocytes, fat cells, type I pneumocytes, pancreatic duct cells, nonstriated duct cells of the sweat gland, nonstriated duct cells of the salivary gland, nonstriated duct cells of the mammary gland, parietal cells of the kidney glomerulus, podocytes of the kidney glomerulus, cells of the thin segment of the loop of Henle, collecting duct cells, duct cells of the seminal vesicle, duct cells of the prostate gland, vascular endothelial cells, synovial cells, serosal cells, squamous cells lining the perilymphatic space of the ear, cells lining the endolymphatic space of the ear, choroid plexus cells, squamous cells of the pia-arachnoid, ciliary epithelial cells of the eye, corneal endothelial cells, ciliated cells having propulsive function, ameloblasts, planum semilunatum cells of (he vestibular apparatus of the ear, interdental cells of the organ of Corti, fibroblasts, pericytes of blood capillaries, nucleus pulposus cells of the intervertebral disc, cementoblasts, cementocytes, odontoblasts, odontocytes, chondrocytes, osteoblasts, osteocytes, osteoprogenitor cells, hyalocytes of the vitreous body of the eye, stellate cells of the perilymphatic space of the ear, skeletal muscle cells, heart muscle cells, smooth muscle cells, myoepithelial cells, red blood cells, platelets, megakaryocytes, monocytes, connective tissue macrophages, Langerhan's cells, osteoclasts, dendritic cells, microglial cells, neutrophils, eosinophils, basophils, mast cells, plasma cells, helper T cells, suppressor T cells, killer T cells, killer cells, rod cells, cone cells, inner hair cells of the organ of Corti, outer hair cells of the organ of Corti, type I hair, cells of the vestibular apparatus of the ear, type II cells of the vestibular apparatus of the ear, type II taste bud cells, olfactory neurons, basal cells of olfactory epithelium, type I carotid body cells, type II carotid body cells, Merkel cells, primary sensory neurons specialised for touch, primary sensory neurons specialised for temperature, primary neurons specialised for pain, proprioceptive primary sensory neurons, cholinergic neurons of the autonomic nervous system, adrenergic neurons of the autonomic nervous system, peptidergic neurons of the autonomic nervous system, inner pillar cells of the organ of Corti, outer pillar cells of the organ of Corti, inner phalangeal cells of the organ of Corti, outer phalangeal cells of the organ of Corti, border cells, Hensen cells, supporting cells of the vestibular apparatus, supporting cells of the taste bud, supporting cells of the olfactory epithelium, Schwann cells, satellite cells, enteric glial cells, neurons of the central nervous system, astrocytes of the central nervous system, oligodendrocytes of the central nervous system, anterior lens epithelial cells, lens fibre cells, melanocytes, retinal pigmented epithelial cells, iris pigment epithelial cells, oogonium, oocytes, spermatocytes, spermatogonium, ovarian follicle cells, Sertoli cells, and thymus epithelial cells, hepatocarcinoma or combinations thereof or cell lines derived therefrom.

20. The composite material according to claim 19 wherein the composite material comprises a constituent element of a biomaterial scaffold.

21. A biomaterial scaffold comprising reconstituted extracellular matrix, polyelectrolyte complex fibers and seeded cells, wherein the extracellular matrix is derived from the same or similar cell type as the seeded cells.

22. The biomaterial scaffold of claim 21, wherein the extracellular matrix is derived from the same cell type as the seeded cells.

23. A method for proliferating, differentiating or maintaining the differentiated phenotype and functions of seeded cells, the method comprising seeding a desired cell type or cell types on a biomaterial scaffold according to claim 1, and culturing said seeded cells under conditions conducive to proliferation, differentiation or maintaining the differentiated phenotype and functions of the seeded cells.
